(19) United States
(12) Patent Application Publication
(10) Pub. No.: US 2009/0155191 A1
(43) Pub. Date: Jun. 18, 2009

(54) Use of chlorhexidine in the prevention of root caries

(76) Inventors: John Marston Symington, Etobicoke (CA); Alison Symington, Toronto (CA); Arnold Richard Ten Cate, Markham (CA); Oliver Ross Perry, Toronto (CA)

Correspondence Address: Rohm & Monsanto, PLC, 12 Rathbone Place, Grosse Pointe, MI, 48230, US

(21) Appl. No.: 12/231,317

(22) Filed: Aug. 28, 2008

Related U.S. Application Data
(63) Continuation of application No. 10/240,494, filed on Jan. 12, 2003, now abandoned.
(60) Provisional application No. 60/194,559, filed on Apr. 3, 2000.

(51) Int. Cl. A61K 6/00; A61Q 11/00; A61C 19/00

(52) U.S. Cl. 424/54; 433/224

(57) ABSTRACT

Topical application of a solution of the antimicrobial chlorhexidine to teeth, particularly including exposed root surfaces, prevents the destruction of exposed cementum and associated exposed enamel at the cementum-enamel junction on tooth root surfaces (root caries) and the inflammation of the gingival tissue. In a method of use, a topical solution containing 10% (w/v) chlorhexidine, 20% (w/v) Sumatra benzoin, and 70% (w/v) ethanol is applied to the appropriate area of the tooth surface, followed immediately by application of a sealant which is a solution containing 29% (w/v) medical-grade polyurethane in 49% (w/v) acetone and 22% (w/v) ethyl acetate. Application of the chlorhexidine and sealant to the tooth cementum and gingival margin of “at risk” older adult patients has significantly reduced the prevalence and incidence of root caries and gingival inflammation.

### RELATIONSHIP TO OTHER APPLICATION(S)

This application is a continuation of U.S. Ser. No. 10/240,494 filed on Jan. 12, 2003, now abandoned. U.S. Ser. No. 10/240,494 is a §371 national stage application of international application no. PCT/IB01/00794 filed on Apr. 3, 2001, which claims the benefit of, U.S. Provisional Application Ser. No. 60/194,559 filed on Apr. 3, 2000.

### BACKGROUND OF THE INVENTION

1. Field of the Invention

The invention relates to the prevention of the destruction of exposed cementum tissue on human tooth root surfaces and the reduction of inflammation of gingival tissues, and more particularly, to the topical application of the antimicrobial agent chlorhexidine to reduce the increment of root caries and to reduce gingival inflammation.

2. Description of the Related Art

Periodontal disease and dental caries are chronic conditions found in the dentition of a significant proportion of the general adult population. There is, therefore, tremendous incentive to develop preventative treatments for these diseases. Both diseases are now generally accepted as bacterial infections found in the dental plaque that forms on teeth and in periodontal pockets.

Periodontal disease refers generally to inflammatory conditions that attack the gingiva and the underlying alveolar bone supporting the teeth. Anaerobic bacteria, such as P. gingivalis and black-pigmented Bacteroides, proliferate in the gingival crevice and produce enzymes, toxins, and noxious metabolites, that accumulate. These bacterial by-products irritate the gingival tissue and initiate a localized inflammation (gingivitis). The inflamed tissue releases enzymes that destroy the collagenous tissue and alveolar bone (periodontitis). If left untreated, this condition will eventually result in loss of the affected tooth.

Dental caries refers to tooth decay or the destruction of tooth surfaces by acids produced by cariogenic bacteria (also known as demineralization). As used in the dental research literature, the term “caries” is used generically to encompass the destruction via demineralization of all tooth surfaces. However, there are two surfaces, or tissues of the tooth, that may be attacked, the enamel on the crown of the tooth and the cementum on the root of the tooth. Moreover, dental research indicates that cariogenic organisms display selectivity as to which tooth surface they will attack and the mix and role of the organisms change as carious lesions progress.

A convenient comparison table outlining the differences between these two tissues is available in Ten Cate, “Oral Histology,” Mosby, Toronto (1998) at page 287. Enamel is a highly mineralized tissue produced by epithelial cells and contains less than 4% organic matter and water. Cementum, on the other hand, is a connective tissue containing approximately 50% organic material that contains significantly less mineralized tissue by weight than enamel. Since cementum tissue differs substantially from the enamel tissue in the crown of a human tooth, the associated demineralization processes for these two tissues differ substantially. For example, the drop in pH necessary to initiate decalcification/demineralization (6.7) in cementum is more than an order of magnitude less than the pH (5.4) necessary to initiate demineralization of enamel. As a result, cementum is more soluble than enamel, and therefore, root lesions progress faster than coronal caries. Moreover, demineralization continues for a longer duration, for each acid challenge, than it does in enamel. Prevention of root caries is important since these lesions are more difficult to restore and lead to tooth loss if untreated. Furthermore, progressive exposure of cementum with age makes it more vulnerable to demineralization associated with certain microorganisms in the oral cavity. As a result, preventative strategies that work on enamel may not be appropriate, or optimum, for cementum, and vice versa. Similarly, strategies to prevent caries in adolescents may not be optimum for preventing caries in adults or elderly.

While the dental research literature implicates Streptococcus mutans in the initiation and progressive demineralization of both enamel and cementum tissues, there is also a significant body of literature that associates Lactobacilli, and most notably Lactobacillus casei, with root caries. See, for example, Beck, Adv. Dent. Res., Vol. 7, No. 1, pp. 42-51 (1993); Faine, et al., Special Care in Dentistry, Vol. 12, No. 4, pp. 177-182 (1992); Hunt, et al., Special Care in Dentistry, Vol. 12, No. 4, pp. 149ff(1992).

In fact, an article by Loesche, et al., Gerodontology, Vol. 16, No. 1 (1999), presented results of a study that demonstrated that S. mutans were significantly associated with coronal surface decay in older individuals (similar in average age to the xerostomia trial described hereinbelow as Clinical Study 1) and that Lactobacilli were significantly associated with root surface decay. The odds/ratio for oral/dental parameters which are significantly associated with decay in elderly individuals, according to the Loesche, et al. study, appears below in Table 1.

TABLE 1
Odds ratio for oral/dental parameters which are significantly
associated with decay in elderly individuals

| factor | Any decay | Enamel surface decay | Cementum decay |
|---|---|---|---|
| S. mutans | 1.12 | 1.12 | |
| Lactobacillus sp. | 1.09 | | 1.24 |

The locus, nature, pathology, etiology, and restorative outcomes of tooth decay, particularly at the root surface, are different in the adult than in the young. In addition to the foregoing, there is a high incidence of caries in patients with salivary hypofunction resulting from chronic medication, systemic disease (e.g., arthritis, lupus, Sjogren's Syndrome) or radiation therapy which conditions are far more prevalent in the adult or older dental patient population. This same population also has a higher incidence of periodontal disease and gingival inflammation.

There is, therefore, a need for a safe and effective preventative treatment for caries, particularly root caries, and gingival inflammation, in “at risk” patient groups, particularly older adults and older adults with salivary hypofunction. This “at risk” population accounts for a minority of dental patients, but a majority of the disease incidence.

Chlorhexidine is a bis biguanide antiseptic and disinfectant that has bactericidal and bacteriostatic action against a wide range of gram-positive and gram-negative bacteria. Chlorhexidine has been used to control the development of caries and is believed to be effective in controlling S. mutans infections. U.S. Pat. No. 4,496,322 describes a dental varnish containing an antimicrobial agent, specifically chlorhexidine acetate, a benzoin gum, and an orally-acceptable solvent that, when applied to teeth, dries to a film, that provides sustained release of the antimicrobial agent. An improvement on this technology was described in U.S. Pat. No. 4,883,534 which further provided a sealing composition, applied to the varnish, to extend the length of the antimicrobial protection provided by the varnish. The aforementioned patents are directed to the reduction of cariogenic bacteria, specifically S. mutans, on tooth surfaces for long periods of time. These patents provide no controlled clinical evidence of the caries or gingival outcomes of this antimicrobial effect, and in particular, these patents provide no indication or reference to the effect on the preservation of cementum tissues from the reduction of bacterial titers in the oral cavity. In fact, based on these patents and the dental research literature, root caries are associated with Lactobacillus, and therefore, one would not expect the application of chlorhexidine dental varnish to have an effect on root caries prevention or control. Nor would one expect the application of chlorhexidine varnish to have an effect on the prevention and control of gingival inflammation resulting from the known periodontal pathogens.

It is, therefore, an object of the invention to provide a method of preventative treatment for caries, particularly root caries.

It is also an object of the invention to provide a method of preventative treatment for root caries and gingival inflammation that is particularly useful for older adults, especially those with saliva hypofunction or xerostomia.

### SUMMARY OF THE INVENTION

The foregoing and other objects, features and advantages are achieved by this invention which provides a method for preventing root caries and/or preventing and controlling gingival inflammation, particularly in patients with active caries on the cementum or cementum-enamel junction or gingival inflammation (gingivitis) or at risk for these conditions. As used herein, the term “preventing root caries” means preventing the destruction of exposed cementum and associated exposed enamel at the cementum-enamel junction on tooth root surfaces.

The method is particularly applicable to the preventive treatment of older patient populations, and patient populations that are “at risk” for root caries and/or gingival inflammation. In one specific embodiment, the method of the present invention was demonstrated to be particularly successful in preventing root caries and gingival inflammation in an “at risk” patient population including the elderly (average age about 60 years old) and, particularly, the elderly (and others) with salivary hypofunction, or xerostomia.

The method comprises contacting the appropriate tooth surface with an effective amount of the antimicrobial agent, chlorhexidine, preferably in a form that provides sustained release of chlorhexidine into the saliva and salivary reservoirs in the oral cavity, such as a dental varnish. The appropriate “tooth surface” for the practice of the present invention includes from the crown down to the gingival margin, and specifically including any exposed dentin between the enamel and the cementum at the cemento-enamel or dentino-enamel junctions.

In the practice of the invention, the tooth surfaces are preferably contacted with a non-toxic, topical solution of chlorhexidine, and preferably up to and including 10% (w/v) chlorhexidine or chlorhexidine salt in solution according to a predetermined dosing schedule. Since it is preferred that chlorhexidine be administered in a form suitable for sustained release of the antimicrobial agent over time, a binder, such as a varnish base or resin, illustratively benzoin gum, is typically employed to create a film-forming solution in an orally-acceptable solvent In a specific preferred embodiment, the topical solution is a varnish comprising 10% (w/v) chlorhexidine, 20% (w/v) Sumatra benzoin, and 70% (w/v) ethanol. This varnish is commercially available in Canada under the trade mark CHLORZOIN (Stage I) from CHX Technologies, Inc., Toronto, Canada.

Of course, the topical solution of chlorhexidine may comprise other non-toxic, biologically acceptable solutions of chlorhexidine and/or salts of chlorhexidine that are suitable for topical application and internal use. Presently known and used salts of chlorhexidine include chlorhexidine acetate, chlorhexidine hydrochloride, and chlorhexidine gluconate. Other orally-acceptable solvents include, but are not limited to, methanol, n-propanol, and isopropanol. The topical solution may also include other ingredients, such as flavorants or flavor masking agents. The topical solution of chlorhexidine is formed by making an admixture of all of the ingredients.

Application of the topical solution of chlorhexidine is most preferably followed by application of a sealant over the chlorhexidine-containing film. In a specific preferred embodiment, the sealant is 29% (w/v) medical-grade polyurethane in 49% (w/v) acetone and 22% (w/v) ethyl acetate. This solution is commercially available in Canada under the trade mark CHLORZOIN (Stage II) from CHX Technologies, Inc., Toronto, Canada. Of course, the sealant can be any solvated non-toxic biodegradable polymer, such as polyurethane or polylactate, that forms a film and is capable of extending retention of the chlorhexidine-containing film at the site of application and/or retarding release of chlorhexidine.

In an alternative embodiment, the sealant further includes other therapeutic components, illustratively a fluoride salt. Since fluoride is known to promote remineralization or construction of enamel surfaces whereas chlorhexidine controls demineralization or destruction of the enamel surfaces, the combination of the chlorhexidine-containing varnish and a fluoride-containing sealant may work in combination to cause a significant beneficial effect. The sustained release of fluoride into the saliva and salivary reservoirs in the oral cavity may actually promote healing of carious lesions. Fluoride-containing sealants may be made by dissolving the desired fluoride salt (e.g., sodium fluoride, sodium monofluorophosphate, or stannous fluoride) in a solvent or mixture of co-solvents. Fluoride-containing sealants are also commercially available, such as DUROFLUOR by Pharmascience, Montreal, Canada (NaF, 50 mg/ml) or FLUOR-PROTECTOR by Ivoclar-Vivadent, Liechtenstein.

The solutions are routinely applied after full dental restoration and after a prophylaxis of all tooth surfaces. The initial application, by either brush or cotton pellet, is the chlorhexidine solution, followed immediately by application of the sealant.

In accordance with another aspect of the present invention, the initial chlorhexidine treatment is repeated on a predetermined dosing schedule. The preferred predetermined dosing schedule is weekly for one month (i.e., for a total of four times) followed by additional treatment at six month intervals for as long as the clinician deems appropriate. Clinical studies have shown that, for xerostomic patients, every 6 months is sufficient to meaningfully reduce caries progression.

An adult dose of solution per treatment ranges from between 400 and 600 ml, thereby depositing about 40 to 60 mg of chlorhexidine in the oral cavity. As noted above, it is preferred in the practice of this invention, that the concentration of chlorhexidine be 10% or less. This amount has been found to be effective for extended periods of time when applied in the preferred dosing schedule. Advantageously, less chlorhexidine reduces adverse side effects such as toxicity, staining, and loss of taste acuity.

In the practice of the invention, the varnish and sealing solutions are stored under refrigerated conditions (preferably ranging from about 2 to 8 degrees Celsius) prior to application to avoid degradation, and can be used for a period of up to 30 months from the date of manufacture.

Although the invention has been described in terms of treatment of humans, particularly humans at risk for root caries and/or periodontal disease, it is to be specifically understood that the invention could find application in the veterinarian field. Therefore, the term “patients” includes mammals, such as cats, dogs, and horses.

### DETAILED DESCRIPTION

### Clinical Results

Due to commercial interest in the use of chlorhexidine, multiple controlled studies were undertaken to determine the efficacy of the use of the antimicrobial drug, chlorhexidine, in controlling caries, and not just in controlling an S. mutans infection. The results of these studies, reported hereinbelow, were unexpected.

In a controlled clinical study reported hereinbelow involving older age groups, selected for participation due to high levels of S. mutans, a solution of chlorhexidine followed by a dental sealant layer showed, surprisingly, that these solutions could meaningfully prevent the incidence of cementum destruction, but produced no comparable results for enamel surfaces. Still, surprisingly, in the study group of older adults (see Clinical Study 1), this treatment controlled both destruction of the cementum and tissue inflammation at the gingival margin.

As indicated, these results were unexpected since the literature associated with the use of chlorhexidine solution and a sealant, including the patent literature as exemplified in U.S. Pat. Nos. 4,496,322 and 4,883,534, reported that these topical applications reduced the key microbial pathogen for caries, S. mutans, to low levels for long periods of time, and hence, it was believed that the enamel surfaces would be protected by this means. The literature has long reported a link between enamel caries and S. mutans infections.

Furthermore, the microorganism Lactobacillus, has been implicated in the dental literature as the key pathogen associated with the demineralization and destruction of cementum. However, it is known that Lactobacillus is resistant to the antimicrobial effects of chlorhexidine. Therefore, it was expected that the cementum would not be afforded meaningful protection from demineralization by the application of chlorhexidine. See, Cleghorn, et al., J. Dent. Res., Vol. 68, pages 1146-1150 (1989); Baker, et al., J. Dent. Res., Vol. 66, No. 6, pp. 1099-1106 (1987).

In addition to the foregoing, it is surprising that the supragingival application of chlorhexidine varnish prevents gingival inflammation since the known periodontopathic organisms responsible for the inflammation reside typically in the subgingival plaque.

In accordance with the principles of the present invention, and for the purposes of the clinical studies reported herein, a topical solution containing 10% (w/v) chlorhexidine, 20% (w/v) Sumatra benzoin, and 70% (w/v) ethanol was applied to the appropriate area of the tooth surface, as defined hereinabove, followed immediately by application of a sealant which was a solution containing 29% (w/v) medical-grade polyurethane in 49% (w/v) acetone and 22% (w/v) ethyl acetate.

This treatment involves the sequential application of the chlorhexidine and then sealant solutions by brush or cotton pellet to the entire surface of the external crown and root of each tooth. In a practical embodiment of the invention, all teeth should be fully restored with no active caries lesions, then cleaned and dried before treatment. The application of the solutions to the soft tissues in the oral cavity should be avoided.

This treatment was repeated weekly for a period of a month. The patient was retreated in accordance with this method at six month intervals thereafter.

Clinical Study 1

In one multi-center controlled study involving 236 adults (average age 58.7 years) with xerostomia (“dry mouth,” measured by unstimulated salivary flow of less than 0.08 ml/min) due to chronic systemic medication, the treatment in accordance with the principles of the invention, as set forth hereinabove, was shown to prevent cementum destruction at a statistically-significant level and to control gingival inflammation at a statistically significant level, different from the control groups. The results are shown in tabular form in Table 2.

Entry criteria for participation in this study included elevated levels of S. mutans (average titer of 11.5×10⁶ cfu per ml saliva), active decay and limited salivary flow (mean unstimulated salivary flow below 0.08 ml per minute) The participants were monitored over a one year period.

TABLE 2
Reduction of Caries Increment on the Enamel and Cementum of
Adult Patients over One Year in a Controlled Clinical Trial

| | | | p Value |
|---|---|---|---|
| | | | (based on a two-sided |
| | | % Reduction | test, last observation |
| | Caries | active vs. | carried forward, intent |
| Group | Increment | placebo | to treat analysis) |
| Active enamel | 1.79 | 14.4% | 0.0644 |
| Placebo enamel | 2.09 | | |
| Active cementum | 0.77 | 40.8% | 0.0249 |
| Placebo cementum | 1.30 | | |

As shown in Table 2, active root caries were reduced by 40% as compared to a placebo (p value of 0.0249). Periodontal health was assessed using conventional techniques. Significant bleeding scores (p=0.015) and plaque accumulation (p=0.003) explains the observed reduction in gingival inflammation. The treatment did not reduce coronal caries at a threshold which is statistically significant.

Other Clinical Studies

In another study, 1265 adolescents (between 11 and 13 in age at baseline) at risk for enamel caries were observed over a three year period. Entry criteria included elevated levels of S. mutans (average titer over 250,000 cfu/ml saliva) and a history of decay. No overall treatment effect was observed between four treatment groups (active, placebo, negative, and positive controls). Most notably, the treatment did not reduce S. mutans levels for time periods described in the scientific literature.

In still another study wherein 210 youngsters (ages 6-7 at baseline) and young adolescents (ages 10-11 at baseline), at risk for enamel caries, were observed over a three year period, similar results were obtained. There was no significant reduction in enamel caries.

Although the invention has been described in terms of specific embodiments and applications, persons skilled in the art may, in light of this teaching, generate additional embodiments without exceeding the scope or departing from the spirit of the invention described herein. Accordingly, it is to be understood that the drawing and description in this disclosure are proffered to facilitate comprehension of the invention, and should not be construed to limit the scope thereof.

What is claimed is:

1. A method for preventing root caries and for preventing and controlling gingival inflammation in a patient population of adults or adults and others who have salivary hypofunction or are at risk for root caries on cementum or associated exposed dentin at a cementum-enamel junction of tooth surfaces and/or gingival inflammation, the method comprising the step of:
contacting tooth surfaces of a patient from the patient population, from a crown surface of the tooth down to a gingival margin, including any exposed dentin between enamel on the crown and cementum, with an effective amount of chlorhexidine once a week for a month (4×) and additionally contacting the tooth surfaces with chlorhexidine at six month intervals thereafter.

2. The method of claim 1 wherein the chlorhexidine is applied as a nontoxic topical solution of a chlorhexidine salt present in an amount up to and including 10% (w/v).

3. The method of claim 2 wherein the chlorhexidine salt is selected from the group consisting of chlorhexidine acetate, chlorhexidine hydrochloride, and chlorhexidine gluconate.

4. The method of claim 2 wherein the topical solution is a film-forming solution that provides sustained release of chlorhexidine.

5. The method of claim 4 wherein the topical solution comprises 10% (w/v) chlorhexidine, 20% (w/v) Sumatra benzoin, and 70% (w/v) ethanol.

6. The method of claim 4 comprising the further step of contacting the tooth surfaces with a sealant.

7. The method of claim 6 wherein the sealant further includes a fluoride salt.

8. The method of claim 1 wherein the effective amount of chlorhexidine is between about 40 to 60 mg per dose.

9. A method for preventing root caries and for preventing and controlling gingival inflammation in a patient population of adults or adults and others who have salivary hypofunction or are at risk for root caries on cementum or associated exposed dentin at a cementum-enamel junction of tooth surfaces and/or gingival inflammation, the method comprising the step of:
a) applying a topical film-forming solution of chlorhexidine to tooth surfaces of a patient from the patient population, from the a crown surface of the tooth down to the a gingival margin, including any exposed dentin between the enamel on the crown and the cementum;
b) applying a sealant over the applied topical solution of chlorhexidine;
c) repeating steps (a) and (b) once a week for one month; and
d) repeating steps (a) and (b) at six month intervals thereafter.

10. The method of claim 9 wherein the film-forming solution of chlorhexidine contains up to and including 10% (w/v) of a chlorhexidine salt.

11. The method of claim 10 wherein the chlorhexidine salt is selected from the group consisting of chlorhexidine acetate, chlorhexidine hydrochloride, and chlorhexidine gluconate.

12. The method of claim 10 wherein the topical solution comprises 10% (w/v) chlorhexidine, 20% (w/v) Sumatra benzoin, and 70% (w/v) ethanol.

13. The method of claim 9 wherein the sealant is a solvated non-toxic biodegradable polymer that forms a film when applied over the applied topical solution of chlorhexidine.

14. The method of claim 13 wherein the sealant further contains fluoride.

15. The method of claim 13 wherein the sealant comprises 29% (w/v) medical-grade polyurethane in 49% (w/v) acetone and 22% (w/v) ethyl acetate.
